(19) United States
(12) Patent Application Publication
(10) Pub. No.: US 2011/0202037 A1
(43) Pub. Date: Aug. 18, 2011

(54) FLUID DELIVERY CATHETER APPARATUS

(76) Inventor: William E. Bolger, Bethesda, MD (US)

(21) Appl. No.: 13/059,640

(22) PCT Filed: Aug. 18, 2009
(86) PCT No.: PCT/US2009/054236

§ 371 Date: Mar. 24, 2011

Related U.S. Application Data
(60) Provisional application No. 61/089,671, filed on Aug. 18, 2008.

(51) Int. Cl. A61M 25/00

(52) U.S. Cl. 604/523

(57) ABSTRACT

An apparatus for delivering a medicated fluid or ointment to a target region of the paranasal sinuses. A flexible dual lumen catheter is provided. A first lumen is releasably attachable to a fluid source. A second lumen is substantially adjacent and parallel to the first lumen, and releasable accepts a deformable member, such as a malleable wire. By bending the deformable member into a desired shape, the flexible second lumen and, in turn, the adjacent flexible first lumen, assume a curvature approximating that of the deformable member. Further, a rigid delivery assembly is provided having a catheter assembly and a plurality of adaptors.

### CROSS-REFERENCE TO RELATED APPLICATION

This application claims the benefit of U.S. Provisional Patent Application No. 61/089,671, filed Aug. 18, 2008, the entirety of which is incorporated herein by reference.

### BACKGROUND OF THE INVENTION

1. Field of the Invention

The present invention relates, in general, to fluid delivery systems for medical applications and, more particularly, to an apparatus capable of delivering sinonasal medications to targeted regions of the paranasal sinuses.

2. General Background of the Invention

Otolaryngology surgeons often encounter patients who continue to have sinusitis following sinus surgery. While certain of these patients respond to oral antibiotics, others commonly do not, or may not tolerate oral antibiotics. Because the patient's sinuses are open following surgery, otolaryngologists have relative access using an endoscope and can instill antibiotics directly into the sinuses. However, suitable instruments to directly instill into the sinuses are not readily available. While a conventional Tuberculin syringe and IV catheter may be used to deliver antibiotics to the more accessible sphenoidal sinuses, the more convoluted cavities of the frontal and maxillary sinuses make direct antibiotic delivery more difficult. Attempts to curve or bend a straight IV catheter are largely unsatisfactory, as the “shape memory” of material from which the IV catheters are typically constructed, together with the fluid force of the antibiotic ointment passing through the IV catheter, both tend to cause such catheters to return towards their prior, linear configuration when attempting to instill sinonasal medications into these sinuses.

Accordingly, it is an object of the present invention to provide an apparatus for instilling medicines, ointments, and catheter wires within the paranasal sinuses.

It is another object of the present invention to provide an apparatus including a catheter capable of being shaped to conform to any required configuration for insertion into the sinus cavities, and to substantially retain such shape.

These and other objects and features of the present invention will become apparent in view of the present specification, drawings and claims.

### BRIEF SUMMARY OF THE INVENTION

The present invention comprises a fluid delivery catheter apparatus. A first lumen having an inlet port, an outlet port, and an elongated body disposed between the inlet port and the outlet port is provided. A second lumen is also provided, with at least a portion of the second lumen being substantially adjacent the first lumen. At least a portion of a deformable member is retainable by the second lumen. In operation, deforming at least a portion of the deformable member to a non-linear shape in turn causes at least a portion of the first lumen to assume a shape approximating the non-linear shape of at least a portion of the deformable member.

The first lumen further includes a connecting region operably attachable to a supply of fluid. This connecting region may comprise, for example, a Luer-Lock connection. The supply of fluid comprises a syringe, or a fluid delivery assembly. The fluid delivery assembly includes a body having a pistol grip, and operably receives a unit dose cartridge. The fluid delivery assembly further includes a delivery actuator causing fluid to be expelled from the unit dose cartridge and into the first lumen. The first lumen may further include an olive tip proximate the outlet port.

At least a portion of at least one of the first lumen and the second lumen are preferably constructed of a substantially flexible material, such as Silastic® or another elastomeric silicone material. The deformable member may comprise, for example, a malleable wire. At least a portion of the wire may be preformed to create a graspable member.

In a further embodiment, the invention provides for a fluid delivery catheter apparatus having a catheter assembly having a tube with a proximal end and a distal end, a first connector coupled to the proximal end and adapted for coupling to a syringe or other fluid delivery system, and a second connector coupled to the distal end. The apparatus further includes an adaptor having a tube with a proximal end and a distal end, the proximal end having a connector for removably coupling to the first connector of the catheter assembly.

The catheter assembly is adapted to accommodate a variety of such adaptors. In this manner, each separate sinus cavity (e.g., maxillary, ethmoid, frontal, sphenoid) can be addressed with a custom adaptor. The fluid delivery catheter apparatus may be used in the pre-surgical and post surgical patient for irrigation and topical delivery of drugs.

### BRIEF DESCRIPTION OF THE SEVERAL VIEWS OF THE DRAWINGS

FIG. 1 of the drawings is an elevated perspective view of the dual lumen catheter and deformable wire of the present invention and showing, in particular, the deformable wire fully inserted into the second lumen and serving to place the catheter into a curved configuration;

FIG. 2 of the drawings is an elevated perspective view of the dual lumen catheter;

FIG. 3 of the drawings is an elevated perspective view of the deformable wire;

FIG. 4 of the drawings is an elevated perspective view of the dual lumen catheter coupled to a syringe;

FIG. 5 of the drawings is an elevated perspective view of the dual lumen catheter coupled to a fluid delivery assembly;

FIG. 6 of the drawings is an elevated perspective view of the dual lumen catheter coupled to an alternative delivery assembly;

FIG. 7 of the drawings is an elevated perspective view of an alternative embodiment of the dual lumen catheter;

FIG. 8 of the drawings is a perspective view of a catheter assembly in accordance with a fluid delivery catheter apparatus of another embodiment of the present invention.

FIG. 8A is an enlarged portion of the catheter assembly of FIG. 8.

FIGS. 9 and 10 of the drawings is a perspective view of an adaptor for use with the catheter assembly of FIGS. 8 and 8A; and

FIGS. 11 and 12 are perspective views of an yet another adaptor for use with the catheter assembly of FIGS. 8 and 8A.

### DETAILED DESCRIPTION OF THE INVENTION

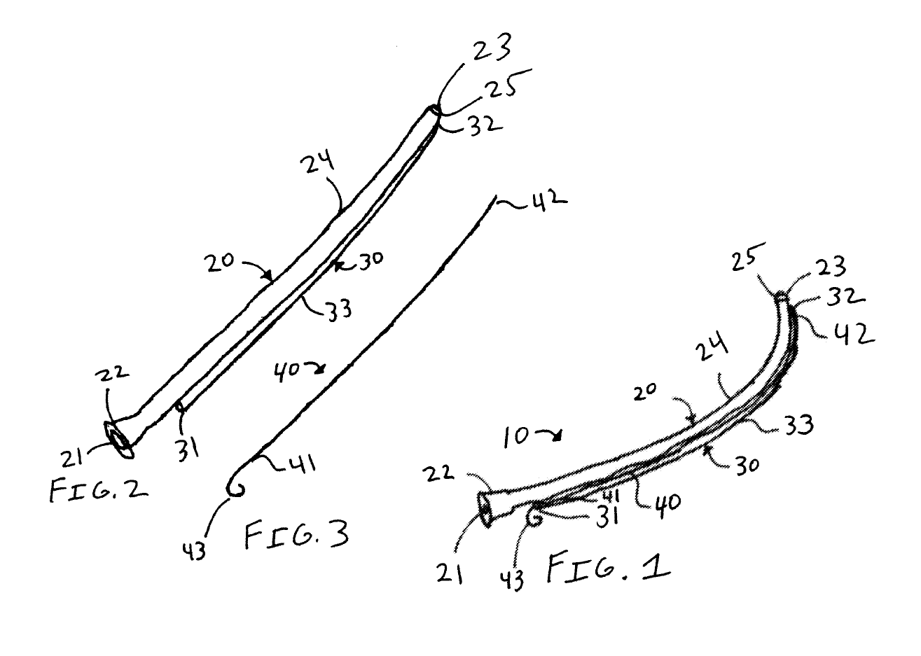

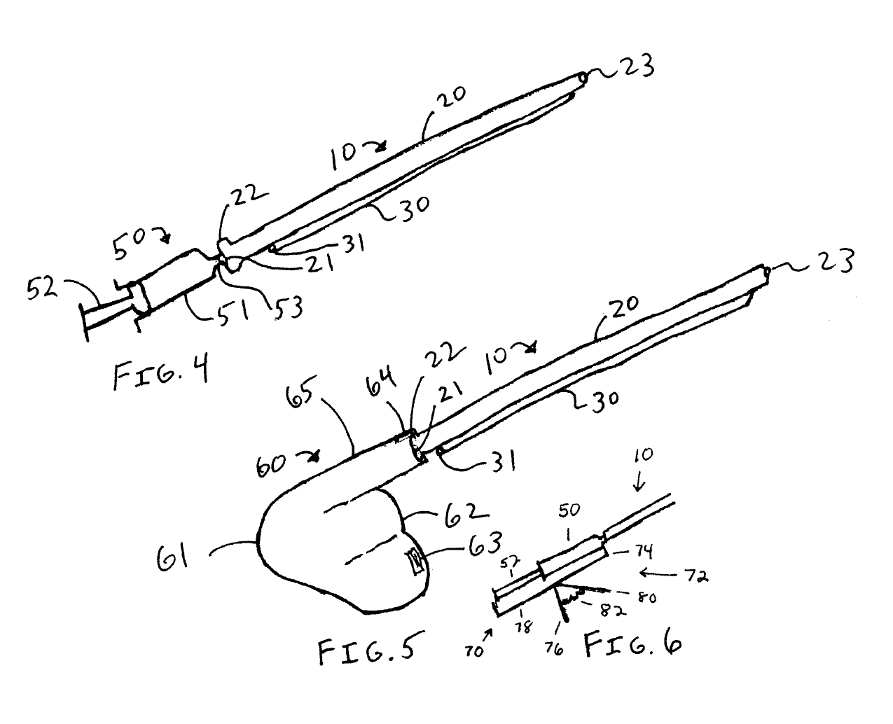

The present fluid delivery catheter 10 is shown in FIGS. 1-3 as comprising first lumen 20, second lumen 30, and deformable member 40. Inlet port 21 is disposed at a proximal end of first lumen 20, and communicates with outlet port 23 at a distal end of first lumen 20. Elongated body 24 of first lumen 20 extends between inlet port 21 and outlet port 23.

Connecting flange 22 disposed at the proximal end of first lumen 20 permits first lumen 20 to be releasably attachable in a substantially sealed, fluid-tight manner to a syringe, delivery assembly, or other pressurized fluid source, as discussed, infra. First lumen 20 optionally includes olive tip 25 proximate distal end 25, to aid in breaking through obstructions and assist in the insertion of catheter 10 into the paranasal sinuses.

Second lumen 30 is parallel and adjacent to an outer surface of first lumen 20, and extends along a substantial length of first lumen 20. Proximal aperture 31 is disposed at a proximal end of second lumen 30, and is sized to accept axial insertion of deformable member 40. Elongated body 33 extends from proximal aperture 31 to closed distal end 32.

Deformable member 40 extends from proximal end 41 to distal end 42. A gripping region 43 or a handle may be disposed at proximal end 41. Gripping region 43 may be preformed by coiling or otherwise shaping a proximal portion of deformable member 40 to facilitate the grasping of deformable member 40 by the surgeon. Alternatively, deformable member 40 may be supplied in linear form, and the surgeon may then manually coil or otherwise shape proximal end 41 to form a gripping region according to preference.

First lumen 10 and second lumen 20 may be constructed, for example, of a substantially flexible material, such Silastic® or other flexible, inert silicone elastomers. Deformable member 40 may be constructed, for example, of a malleable wire or other material suitable to being bended or otherwise shaped by the physician.

As shown in FIG. 1, deformable member 40 may be partially or almost entirely inserted into second lumen 30, by axial insertion and advancement of distal end 42 of deformable member 40 through proximal aperture 31 of second lumen 30. As shown in FIG. 1, deformable member 40 and, in turn, the associated length of second lumen 30 and first lumen 20, may be curved, angled, or bent into any of a wide variety of configurations, as the relatively flexible construction of first lumen 20 and second lumen 30 enable them to assume whatever shape deformable member 40 is bent or otherwise deformed into. In operation, deformable member 40 may later be retracted from second lumen 30 through proximal aperture 31 to permit further advancement of fluid delivery catheter 10 within the targeted paranasal sinus cavity.

In one alternative embodiment of the present invention, second lumen 30 may be carried with the interior channel of first lumen 20, instead of being disposed adjacent an outer surface. In another alternative embodiment, second lumen 30 may be integrally formed with first lumen 20, such that second lumen 30 forms an interior channel or passage running along and carried within the material forming first lumen 20. In yet another alternative embodiment, proximal aperture 31 of second lumen 30 may instead be sealed, permanently carrying deformable member 40 therewithin. Moreover, in other alternative embodiments, deformable member 40 may instead be integrally formed within a length of first lumen 20, or affixed to an outer or inner surface of first lumen 20, thus eliminating any requirement for second lumen 30.

As shown in FIG. 4, fluid delivery catheter 10 may be coupled to syringe 50, as one potential source of pressurized fluid. In particular, connecting flange 22 may be securely coupled to connecting region 53 of syringe 50, causing inlet port 21 and, in turn, the interior of first lumen 20, to be in fluid communication via the substantially fluid-tight seal that is formed upon connection. The connection of fluid delivery catheter 10 to syringe 50 may employ, for example, a conventional Luer-Lock connection. Plunger 52 may then be advanced into barrel 51 of syringe 51, causing antibiotics or other sinonasal medications in fluid or ointment form, contained within barrel 51, to be expelled through connecting region 53 of syringe 50, through inlet port 21 of first lumen 30, and finally our of outlet port 23 to the targeted region of the patient.

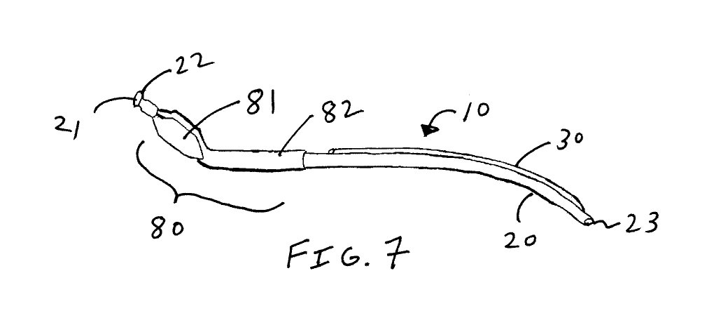

As shown in FIG. 5, fluid delivery catheter 10 may instead be coupled to fluid delivery assembly 60. Fluid delivery assembly 60 includes body 61 having pistol grip region 62 and delivery actuator, or button 63. Connecting region 65 permits connecting flange 22 of first lumen 30 to be securely coupled to connecting region 64, causing inlet port 21 and, in turn, the interior of first lumen 20 to be in fluid communication with a unit dose cartridge releasable held within barrel region 65 of fluid delivery system 60. The connection of fluid delivery catheter 10 to fluid delivery assembly 60 may employ, for example, a conventional Luer-Lock connection. The pressing of delivery button 63 causes antibiotics or other sinonasal medications in fluid or ointment form, contained within the unit dose cartridge, to be expelled through connecting region 64 of fluid delivery assembly 60, through inlet port 21 of first lumen 30, and finally our of outlet port 23 to the targeted region of the patient.

As shown in FIG. 6, the fluid delivery catheter 10 may instead be coupled to a fluid delivery assembly 70. The fluid delivery assembly 70 includes a combination syringe 50 and syringe holder and actuator mechanism 72. The mechanism 72 includes a syringe retaining bracket 74 with depending handle 76, syringe actuation bracket 78, and a trigger 80 biased by a spring 82. The syringe actuation bracket 78 includes a plurality of notches (not shown) extending along the length of the bracket 78. The upper end portion of the trigger 80 includes a flange (not shown). The flange is adapted to engage any one of the notches in the bracket 78.

The fluid delivery assembly 70 allows the user to control the unit dosage to the patient. The spring biased trigger 80 pivotally coupled to the bracket 74 and engages the syringe actuation bracket 78. Pressing the trigger 80 causes the trigger 80 to pivot such that the flange engages one of the notches and advances the syringe actuation bracket 78 forward, urging the plunger 52 forward. Subsequent release of the trigger 80 allows the trigger 80 to pivot back to the spring biased position with the flange ready for engagement with an adjacent notch. Repeated activation of the trigger 80 causes incremental advancement of the plunger 52 allowing controlled dosage to the treatment location.

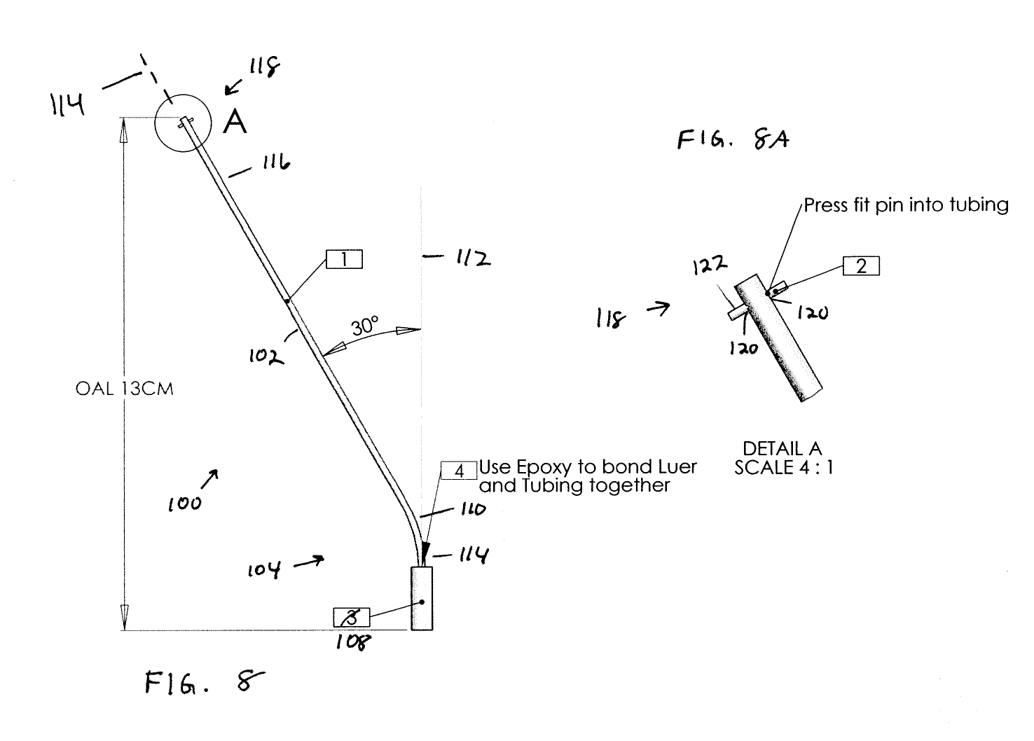

An alternative embodiment of the fluid delivery catheter of the present invention is shown in FIG. 7. In this embodiment, fluid delivery catheter 10 includes substantially rigid region 80 at the proximal end of the catheter, surrounding first lumen 20 and serving to facilitate grasping of fluid delivery catheter 10, particularly when attaching the catheter to a syringe or other source of fluid. In particular, rigid region 80 includes shaft portion 82 and grasping member, or grasping region 81 and may comprise, for example, plastic or other rigid material, and may be over-molded or otherwise formed or disposed about the proximal end of first lumen 20.

FIGS. 8-12 shows yet a further embodiment of a fluid delivery catheter apparatus or system. In this embodiment, the dual lumen embodiment is omitted in favor of a rigid apparatus.

FIG. 8 shows a catheter assembly 100. In one embodiment, the catheter assembly 100 includes a tube 102 having proximal end 104 and a distal end 106 and a lumen extending therebetween. In one embodiment, the tube 102 is made of stainless steel. However, it will be apparent that other material may be used. A connector 108 is secured to the proximal end 104. The connector 108 may be secured to the tube 102 via a medical grade epoxy, for example. Other means of securing the connector 108 to the tube 102 will be apparent. The connector 108 is adapted for coupling a syringe (not shown) to the catheter assembly 100. For example, the connector 108 may be a female Luer type connector for coupling to a corresponding male Luer type connector (not shown) of a syringe.

It can be seen from FIG. 8 that the tube 102 includes a portion 110 having a bend. In one embodiment, the bend portion 110 offsets the longitudinal axis 112 of a proximal section 114 of the tube 102 by 30 degrees from the longitudinal axis 114 of a distal section 116. As it will be appreciated, the amount of degrees of the offset may vary. The amount of the offset is selected to enhance the handling of the catheter assembly during use.

FIG. 8A shows an enlarged view of the distal end 106. The distal end 106 includes a connector 118. In one embodiment, the connector 118 includes a pair of aligned openings 120 in the wall of the tube 102. A pin 122 is press fit into the tube 102 as shown in FIGS. 8 and 8A. In one embodiment, the pin 122 is also made of stainless steel. The connector 118 is provided for coupling to a variety of adaptors as described in greater detail below.

In one embodiment, the tube 102 is about 5 inches in length, with an outside diameter of 0.105 inches and an inside diameter of 0.074 inches. The pin 122 is about 0.154 inches in length with a diameter of 0.02 inches.

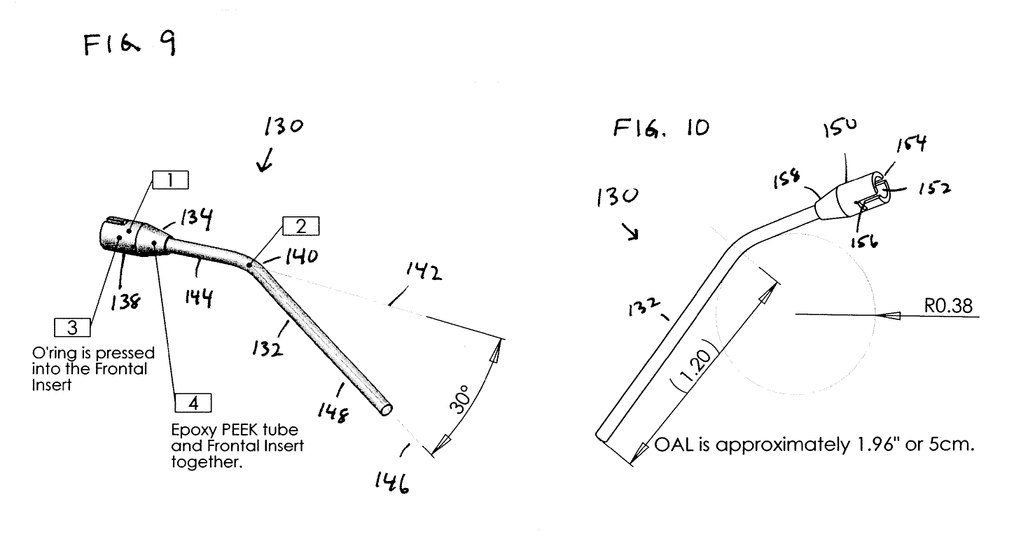

FIG. 9 shows an adaptor 130 for coupling to the catheter assembly 100. The adapter 130 includes a tube 132 having proximal end 134 and a distal end 136 and a lumen extending therebetween. In one embodiment, the tube 132 is made of PTFE, silicon or PEEK. However, it will be apparent that other material may be used. A connector 138 is secured to the proximal end 134. The connector 138 may be secured to the tube 132 via a medical grade epoxy, for example. Other means of securing the connector 138 to the tube 132 will be apparent. The connector 138 is adapted for coupling to the connector 118 of the catheter assembly 100.

It can be seen from FIG. 9 that the tube 132 includes a portion 140 having a bend. In one embodiment, the bend portion 140 offsets the longitudinal axis 142 of a proximal section 144 of the tube 132 by 30 degrees from the longitudinal axis 146 of a distal section 148. As it will be appreciated, the amount of degrees of the offset may vary. The amount of the offset is selected to enhance the handling of the adaptor 130 during use.

FIG. 10 shows another view of the adaptor 130 of FIG. 9. FIG. 10 shows the connector 138 includes a sleeve portion 150 having an opening 152. The sleeve portion 150 further includes two corresponding longitudinally extending slots 154 which open at the opening 152. The sleeve portion 150 further includes two corresponding lateral slots 156. The lateral slots 156 extend from the respective slot 154 in a direction substantially perpendicular to the longitudinally extending slots 154. However, the lateral slots are curved or otherwise extend slightly from the respective slots 154 in a direction towards the opening 152 in a manner as will be understood.

The connector 138 further includes a shoulder portion 158. The shoulder portion 158 includes an end wall in contact with the sleeve portion 150. The end wall includes a bore which is in fluid communication with the lumen of the tube 132 and the opening of the sleeve portion. An O-ring is located within the opening of the sleeve portion and in engagement with the end wall. While the end wall and bore of the connector in FIGS. 9 and 10 are not shown, such features are further described below.

It will be appreciated that the connector 118 may be coupled to the connector 138, with the resilient action of the O-ring and the interacting nature of the lateral slots 156 and pins 122 will retain the connector 118 and 138 in sealed engagement.

In one embodiment, the tube 132 has an outside diameter of 0.062 inches. The O-ring has an inside diameter of 0.098 inches.

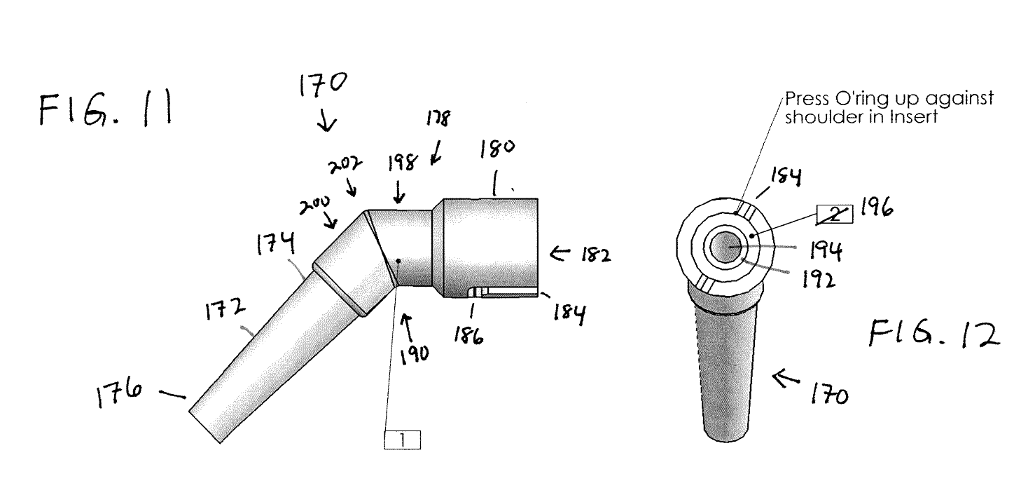

FIG. 11 shows adaptor 170 for coupling to the catheter assembly 100. The adapter 170 includes a tube 172 having proximal end 174 and a distal end 176 and a lumen extending therebetween. In one embodiment, the tube 172 is made of PTFE or silicon. However, it will be apparent that other material may be used. A connector 178 is secured to the proximal end 174. The connector 178 may be secured to the tube 172 via a medical grade epoxy, for example. Other means of securing the connector 178 to the tube 172 will be apparent. The connector 178 is adapted for coupling to the connector 118 of the catheter assembly 100.

FIG. 11 shows the connector 138 includes a sleeve portion 180 having an opening 182. The sleeve portion 180 further includes two corresponding longitudinally extending slots 184 which open at the opening 182. The sleeve portion 180 further includes two corresponding lateral slots 186. The lateral slots 186 extend from the respective slot 184 in a direction substantially perpendicular to the longitudinally extending slots 184. However, the lateral slots 186 are curved or otherwise extend slightly from the respective slots 184 in a direction towards the opening 182 in a manner as will be understood.

The connector 170 further includes a shoulder portion 190. As seen in FIG. 12, the shoulder portion 190 includes an end wall 192 in contact with the sleeve portion 180. The end wall 192 includes a bore 194 which is in fluid communication with the lumen of the tube 172 and the opening 182 of the sleeve portion 180. An O-ring 196 is located within the opening 182 of the sleeve portion 180 and in engagement with the end wall 192.

It will be appreciated that the connector 118 may be coupled to the connector 170, with the resilient action of the O-ring 196 and the interacting nature of the lateral slots 186 and pins 122 will retain the connector 118 and 170 in sealed engagement.

It can be seen from FIG. 11 that the shoulder portion 190 includes a first portion 198 and a second portion 200 which form a bend portion 202. The bend portion 202 offsets the longitudinal axis of the tube 172 from the longitudinal axis of the sleeve portion 180. As it will be appreciated, the amount of degrees of the offset may vary. The amount of the offset is selected to enhance the handling of the adaptor 170 during use.

1. A fluid delivery catheter apparatus comprising:
a first lumen having an inlet port, an outlet port, and an elongated body disposed between the inlet port and the outlet port;
a second lumen, at least a portion of the second lumen being substantially adjacent the first lumen;
a deformable member, at least a portion of the deformable member being retainable by the second lumen;
whereby deforming at least a portion of the deformable member to a non-linear shape in turn causes at least a portion of the first lumen to assume a shape approximating the non-linear shape of at least a portion of the deformable member.

2. The invention according to claim 1, wherein the first lumen further includes a connecting region operably attachable to a supply of fluid.

3. The invention according to claim 2, wherein the connecting region comprises a Luer-Lock connection.

4. The invention according to claim 2, wherein the supply of fluid comprises a syringe.

5. The invention according to claim 2, wherein the supply of fluid comprises a fluid delivery assembly.

6. The invention according to claim 5, wherein the fluid delivery assembly includes a body having a pistol grip.

7-8. (canceled)

9. The invention according to claim 1, wherein at least a portion of at least one of the first lumen and the second lumen are constructed of a substantially flexible material.

10. The invention according to claim 9, wherein the substantially flexible material is an elastorneric silicone material.

11. (canceled)

12. The invention according to claim 1, wherein the deformable member comprises a malleable wire.

13. The invention according to claim 12, wherein at least a portion of the malleable wire is preformed to create a graspable member.

14. The invention according to claim 1, wherein the first lumen further includes an olive tip proximate the outlet port.

15. The invention according to claim 1, further comprising a substantially rigid region associated with the first lumen.

16. The invention according to claim 15, wherein the substantially rigid region substantially surrounds at least a portion of a proximal end of the first lumen.

17. The invention according to claim 15, wherein at least a portion of the substantially rigid region comprises a grasping region.

18. A fluid delivery catheter apparatus comprising:
a catheter assembly having a tube with a proximal end and a distal end, a first connector coupled to the proximal end and adapted for coupling to a syringe or other fluid delivery system, and a second connector coupled to the distal end; and
an adaptor having a tube with a proximal end and a distal end, the proximal end having a connector for removably coupling to the first second connector of the catheter assembly.

19. The apparatus of claim 18, wherein a substantial portion of the tube extends at an angle from the longitudinal axis of the first connector of the catheter assembly, preferably at an angle of approximately 30 degrees.

20. The apparatus of claim 18, wherein a substantial portion of the tube extends at an angle from the longitudinal axis of the adaptor connector.

21. The apparatus of claim 18, wherein the first connector is a Luer type connector for coupling to a syringe or other fluid delivery system.

22. The apparatus of claim 18, wherein the second connector includes a pin extending through the walls of the tube at a right angle to the longitudinal axis of the tube.

23. The apparatus of claim 18, wherein the catheter assembly and the adaptor are rigid.

24. (canceled)
